(19) United States
(12) Patent Application Publication
(10) Pub. No.: US 2009/0275982 A1
(43) Pub. Date: Nov. 5, 2009

(54) DEVICE FOR TREATING VERTEBRAE, INCLUDING AN INTERSPINOUS IMPLANT

(76) Inventor: Jean Taylor, Cannes (FR)

Correspondence Address: MEDTRONIC;Attn: Noreen Johnson - IP Legal Department, 2600 Sofamor Danek Drive, Memphis, TN, 38132, US

(21) Appl. No.: 12/296,958

(22) PCT Filed: Apr. 3, 2007
(86) PCT No.: PCT/IB2007/000982

§ 371 Date: Apr. 28, 2009

Related U.S. Application Data
(60) Provisional application No. 60/791,906, filed on Apr. 14, 2006.

(30) Foreign Application Priority Data
Apr. 13, 2006 (FR) 06 03266

(51) Int. Cl. A61B 17/70

(52) U.S. Cl. 606/249

(57) ABSTRACT

This device (10) includes an interspinous implant (1) having a flexible structure, capable of being inserted between the spinous processes (100) of two vertebrae, the areas (Z) of these respective spinous processes (100) bearing against the implant (1) defining a longitudinal axis (L) of the implant (1). According to the invention, the device includes at least one cerclage (15) capable of being adjustably engaged around the implant (1), at a centre section (4) of this implant (1), in a plane (P) perpendicular to said longitudinal axis (L); this cerclage (15) is made of a material that is inextensible in the circumferential direction of this cerclage (15), so that it makes it possible to restrain deformation of the implant (1) at the centre section (4), within the entire said plane (P), during exertion on this centre section (4) of opposing compressive forces exerted by the spinous processes of the vertebrae during a posterior movement of the spinal column.

This invention relates to a device for treating vertebrae, including an interspinous implant.

In the document No. WO 99 42051, in the name of the applicant, it is known to produce a flexible interspinous implant, capable of cushioning the posterior movement of the vertebrae during extension of the spinal column. This implant is substantially in the shape of an “H.” i.e., it includes an upper recess and a lower recess capable of receiving the spinous processes of the two vertebrae; it is formed from a block of flexible material, made of silicone in particular, possibly contained inside a casing made of a woven material, made of polyester in particular.

This implant is satisfactory in its usage, constituting an alternative to the other, more invasive methods for treating vertebrae, in particular the interpedicular osteosynthesis methods consisting of installing a rigid device with pedicular screws and connecting rods joining together the treated vertebrae.

However, it appears that, in some cases, particularly in the case of patients who are strong or have localised compensatory hyperlordosis, the existing implant has an insufficient cushioning capability. Installation in such patients of the larger size implant existing within the range of available implants is not a satisfactory solution since it is liable to cause too large of a distraction of the vertebrae, leading to excessive anterior disc pressure.

The documents U.S. Pat. No. 5,496,318, FR 2 844 179 and FR 2 730 156 disclose various other interspinous implant systems, which do not however make it possible to remedy the above-mentioned problem. In particular, the document U.S. Pat. No. 5,496,318 discloses an implant that is necessarily rigid, taking into consideration the connecting sections of the two portions of the implant (the recommended material for the implant can in particular be high-density polyethylene).

This invention aims to remedy the aforesaid problem of the insufficient cushioning capability of the implant.

In a manner known per se, the device in question includes an implant having a flexible structure, capable of being inserted between the spinous processes of two vertebrae, the areas of these respective spinous processes bearing against the implant defining a longitudinal axis of the implant.

According to the invention, the device includes at least one cerclage capable of being adjustably engaged around the implant, at a centre section of this implant, in a plane perpendicular to said longitudinal axis; this cerclage is made of a material that is inextensible in the circumferential direction of this cerclage, so that it makes it possible to restrain deformation of the implant at the centre section, within the entire said plane, during exertion on this centre section of opposing compressive forces exerted by the spinous processes of the vertebrae during a posterior movement of the spinal column.

As a matter of fact, in certain clinical anatomic situations, the inventor was able to observe an insufficient cushioning capability of the existing implant, resulting from a lateral creep of the implant material under the effect of said compressive forces. The cerclage according to the invention makes it possible to impede this lateral deformation and to thus preserve a sufficient cushioning capability at the centre section of the implant, even in the case of strong compressive forces, such as may exist in patients who are strong or suffering from hyperlordosis.

This cerclage can be put into place before inserting the implant into the patient, or can be put into place on the implant after the latter has been placed on the spinal column of the patient. In the second case, the implant retains a significant degree of flexibility, enabling it to be inserted and put into place in a minimally invasive manner.

The cerclage could have a reduced width, e.g., a circular cross-section. Preferably, however, the cerclage has a flattened cross-section forming a planar surface for bearing against the implant.

The risk of a shearing effect of the implant material on either side of the cerclage is thereby greatly reduced, and the bearing surface of the cerclage against the implant is better distributed.

In the same aim, the cerclage may include rounded edges on said planar surface for bearing.

The cerclage preferably includes an increased width near at least one of its portions intended to be applied against the lateral areas of the implant.

This width increase makes it possible to strengthen the restraining function of the cerclage and to increase the contact surface between the cerclage and the implant, in order to eliminate any shearing risk of the implant material.

Alternatively, and for the same purpose, the device can include at least one bearing plate made of a rigid and heavy-duty material, intended to be inserted between the implant and the cerclage.

Said increased width portion of the cerclage, or said plate, can have a dimension such that it is capable of widely wrapping around a corresponding lateral area of the implant, i.e., of extending not only over this lateral area but also partially over the posterior and anterior faces of the implant.

This wrap-around shape furthers the lateral restraint of the implant. In the case of said plate, it also enables a certain degree of retention of this plate on the implant, facilitating the installation of the device.

On its outside face, at least one plate advantageously includes a recess for receiving the cerclage.

This recess enables the cerclage to be wedged in relation to the plate, ensuring their positioning in relation to each other and preventing a risk of the cerclage slipping over the course of time, in relation to the implant.

The plate and/or the cerclage can have shapes enabling assembly of this plate and this cerclage, in particular by snapping or force-fitting together, with a self-locking effect.

This assembly contributes to facilitating the installation of the cerclage and the plate or plates on the implant.

The cerclage can consist of a continuous strip of material inserted over the implant, or can consist of a strip of material whose ends are joined together after installation. In this case, these ends can be joined together by any means, in particular by tying, welding, suturing, with a non-return cam of the type described in the document WO 94 26192, by crimping, or by deforming and/or folding the material.

The invention will be well understood, and other characteristics and advantages of it will become apparent, with reference to the appended schematic drawing, which, for non-limiting illustrative purposes, shows several possible embodiments of the device to which it relates.

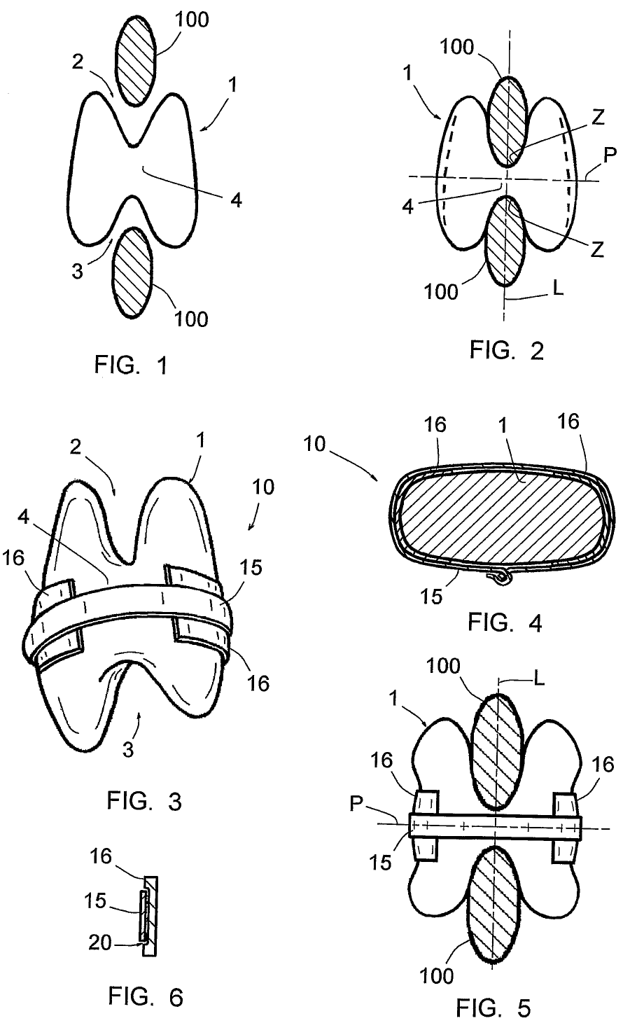

FIG. 1 is a posterior view of an existing implant, the spinous processes of the vertebrae being shown as a sectional view and in the lordosis position;

FIG. 2 is a view similar to FIG. 1, the spinous processes of the vertebrae being shown in extended position of the spinal column, i.e., exerting opposing pressures on the implant;

FIG. 3 is a perspective view of the device according to the invention, according to one embodiment;

FIG. 4 is a view of this device according to another embodiment, as a sectional view of the centre section of an implant that includes this device;

FIG. 5 is a perspective view of the device, similar to FIG. 2, and

FIG. 6 is an enlarged-scale detail view of another embodiment of the device.

FIGS. 1 and 2 show an existing flexible interspinous implant 1, capable of being inserted between the spinous processes 100 of two vertebrae so as to be able to cushion the posterior movement of these vertebrae during extension of the spinal column. The implant I is substantially in the shape of an “H,” i.e., it includes an upper recess 2 and a lower recess 3 capable of receiving the spinous processes 100; it is formed from a block of flexible material, made of silicone in particular, possibly contained inside a casing made of a woven material, made of polyester in particular.

In the event of a posterior movement of the spinal column, as is shown in FIG. 2, the spinous processes 100 exert opposing pressures on the centre section 4 of the implant 1, which can be significant in patients who are strong or who have localised compensatory hyperlordosis. Such pressures produce a lateral creep of the implant 1 material, resulting in a thinning of the centre section 4 that does not enable a sufficient capability for cushioning the posterior movements of the vertebrae.

The areas Z of the respective spinous processes 100 bearing against the implant 1 define a longitudinal axis L of the implant 1, and a plane P perpendicular to this longitudinal axis.

FIGS. 3 to 6 show a device 10 for treating vertebrae, including an interspinous implant 1 such as the one cited above. The device further includes a restraining cerclage 15 and two lateral bearing distribution plates 16.

The cerclage 15 is made of a stretchable material, in particular a synthetic or metallic material. It has a flattened cross-section forming a planar surface for bearing against the implant 1.

As shown in FIGS. 3 to 5, this cerclage 15 is intended to be adjustably engaged around said centre section 4 of the implant 1, in a plane perpendicular to the longitudinal median plane of the implant, i.e., the plane of symmetry of the implant 1 passing through the bottoms of the recesses 2, 3. The cerclage 15 can consist of a continuous strip, as shown in FIGS. 3 or 5, and is put into place before inserting the implant 1 into the body of the patient, or can be put into place on the implant 1 after the latter has been placed on the spinal column of the patient. In the this second case, the cerclage 15 can, in particular, be made of a metallic material and, as shown in FIG. 4, one of its ends can include a hole into which the other of its ends can be inserted, this other end then being folded over itself and crimped in order to ensure closure of the cerclage 15.

Each plate 16 is made of a rigid and heavy-duty material, in particular a synthetic or metallic material. As shown more particularly in FIG. 4, when seen from a profile view, it has a “C” shape capable of widely wrapping around a corresponding lateral area of the implant 1, i.e., of extending not only over this lateral area but also partially over the posterior and anterior faces of the implant 1.

As seen in the figures, the two plates 16 are intended to be inserted between the implant 1 and the cerclage 15 and function to increase the contact surface between the cerclage 15 and the implant 1.

FIG. 6 shows that, on its outside face, at least one plate 16 can include a recess 20 for receiving the cerclage 15, making it possible to wedge the cerclage 15 in relation to the plate 16. This wedging ensures the positioning of the cerclages 15 and the plate 16 in relation to each other and prevents a risk of this cerclage 15 slipping over the course of time, in relation to the implant 1.

As can be understood with reference to FIG. 5, and by comparison with FIG. 2, during the posterior movement of the spinal column, leading to a drawing together of the spinal processes 100 of the two vertebrae treated, the cerclage 15 makes it possible to restrain the lateral deformation of the implant 1 at the centre section 4. This restraint makes it possible to impede the lateral deformation that the implant 1 would otherwise experience, and to thus preserve a sufficient cushioning capability at the centre section 4, even in the case of strong compressive forces, such as can those that can exist in patients who are strong or who suffer from hyperlordosis.

The plates 16 strengthen the restraining function of the cerclage 15 and make it possible to increase the contact surface between the cerclage 15 and the implant 1, in order to eliminate any shearing risk of the implant material. Their wrap-around shapes further the lateral restraint of the implant 1 and enable a certain degree of retention of these plates 16 on the implant 1, facilitating the installation of the device.

As is apparent from the preceding, the invention provides a device for treating vertebrae whose interspinous implant has a sufficient cushioning capability in every instance, in particular in the case of patients who are large or who have localized compensatory hyperlordosis.

It is obvious that the invention is not limited to the embodiment described above for illustrative purposes, but that it extends to any embodiment covered by the appended claims.

1.-10. (canceled)

11. A device for treating vertebrae, comprising:
a flexible implant capable of being inserted between adjacent spinous processes; the implant having recesses for receiving the spinous processes and a central portion disposed therebetween;
at least one constricting band adjustably engaged around the implant central portion and disposed generally in a plane oriented generally perpendicular to an axis extending through the recesses;
the constricting band formed of a material that is inextensible along the length of the band so that the band restrains deformation of the implant central portion in the plane when the central portion is subjected to compressive forces exerted by the spinous processes during spinal extension.

12. The device of claim 11 wherein the band has a flattened cross-section forming a planar surface for bearing against the implant.

13. The device of claim 12 wherein the band has rounded edges on said planar surface for bearing.

14. The device of claim 11 wherein the implant has first and second lateral sections extending through the plane and disposed in laterally spaced relation to the recesses; wherein the band has an increased width portion proximate at least one of the lateral sections.

15. The device of claim 14 wherein said increased width portion extends at least partially around a posterior or an anterior face of the implant when the implant is inserted between the spinous processes such that the recesses receive the spinous processes.

16. The device of claim 11 further comprising a rigid bearing plate disposed between the implant and the band.

17. The device of claim 16 wherein said bearing plate extends at least partially around a posterior or an anterior face of the implant when the implant is inserted between the spinous processes such that the recesses receive the spinous processes.

18. The device of claim 16 wherein the plate comprises a recess on an outboard face thereof for receiving the band.

19. The device of claim 16 wherein the plate and the band are locked together to prevent relative movement therebetween in a direction normal to the plane.

20. The device of claim 11 wherein the band forms an endless loop.

21. The device of claim 11 wherein the band has ends that are joined together.
